(19) United States
(12) Plant Patent Application Publication
(10) Pub. No.: US 2007/0163016 P1
(43) Pub. Date: Jul. 12, 2007

(54) Raspberry plant named 'Driscoll Pacifica'

(76) Inventors: Carlos Fear, Aptos, CA (US); Rick Harrison, Aptos, CA (US)

Correspondence Address: JONES DAY, 222 EAST 41ST ST, NEW YORK, NY, 10017, US

(21) Appl. No.: 11/327,134

(22) Filed: Jan. 6, 2006

(51) Int. Cl. A01H 5/00

(52) U.S. Cl. PLT/204

(57) ABSTRACT

The present invention relates to a new and distinct cultivar of raspberry plant named ‘Driscoll Pacifica’. The new cultivar is distinguished from other raspberry cultivars by its fruit firmness, consistent large fruit size and high yield. The new cultivar is distinguished from its parent by having better flavored fruit and larger fruit.

### 1. LATIN NAME OF THE GENUS AND SPECIES OF THE PLANT CLAIMED

The variety is botanically identified as Rubus idaeus L. subgenus Rubus.

### 1.2 VARIETY DENOMINATION

The Raspberry variety denomination is ‘Driscoll Pacifica’.

### 2. BACKGROUND OF THE INVENTION

This invention relates to a new cultivar of raspberry called ‘Driscoll Pacifica’. The new cultivar was developed from the hybridization of the selection ‘N234.1’ (an unpatented variety) as the seed parent with the selection ‘Q471.6’ (an unpatented variety) as the pollen parent. The parents were crossed in 1996; whereafter fruit and seed were collected to produce seedlings for field planting in Carpenteria, Calif. in 1997. The new cultivar was selected from these seedlings in 1997 for its large, firm and good flavoured fruit. The new cultivar has been asexually propagated by in vitro shoot tip culture, root sucker division and root cuttings at the Cassin Ranch in Santa Cruz County, Calif. and has been shown to maintain the desired and distinguishing characteristics after propagation over several generations.

### 3. SUMMARY OF THE INVENTION

The present invention provides a new and distinct cultivar of red raspberry plant named ‘Driscoll Pacifica’. The cultivar is botanically identified as Rubus idaeus L. The ‘Driscoll Pacifica’ red raspberry plant produces a primocane crop which begins in late June-mid July and continues until late October. The floricane crop begins in early May and continues until mid July. Both the primocane and floricane yields are high relative to other comparable varieties. The fruit of ‘Driscoll Pacifica’ is notably quite firm and good flavor. The fruit of ‘Driscoll Pacifica’ separates easily from its receptacle.

### 4. BRIEF DESCRIPTION OF THE DRAWINGS

The accompanying photographs show typical specimens of the primocane fruit, leaves and shoot of the new cultivar, in color as nearly true as it is reasonably possible to make in color illustrations of these characteristics.

FIG. 1 is a photograph of ‘Driscoll Pacifica’ primocane flower and fruit in various stages of development.

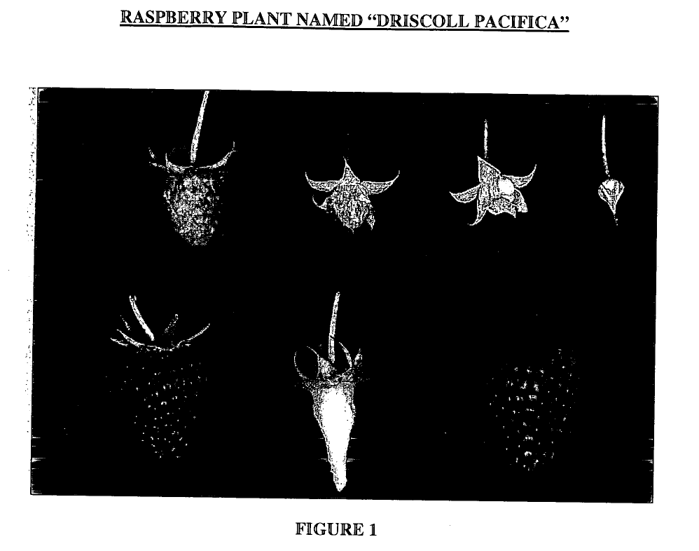

FIG. 2 is a photograph of ‘Driscoll Pacifica’ primocane leaves showing upper and lower leaf surfaces.

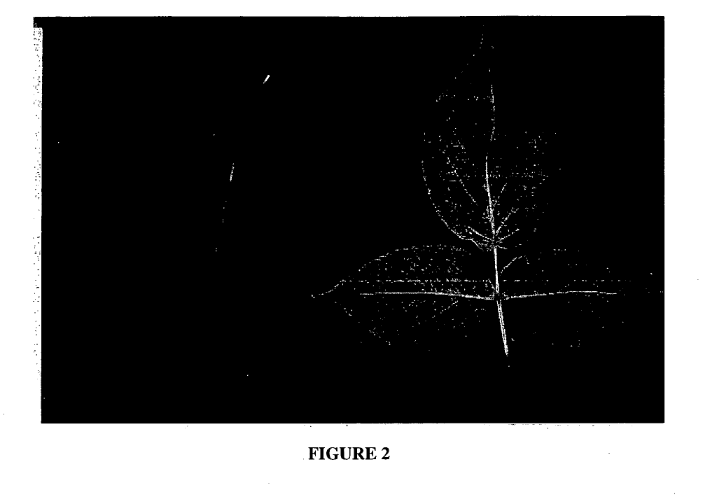

FIG. 3 is a photograph of ‘Driscoll Pacifica’ primocane shoot.

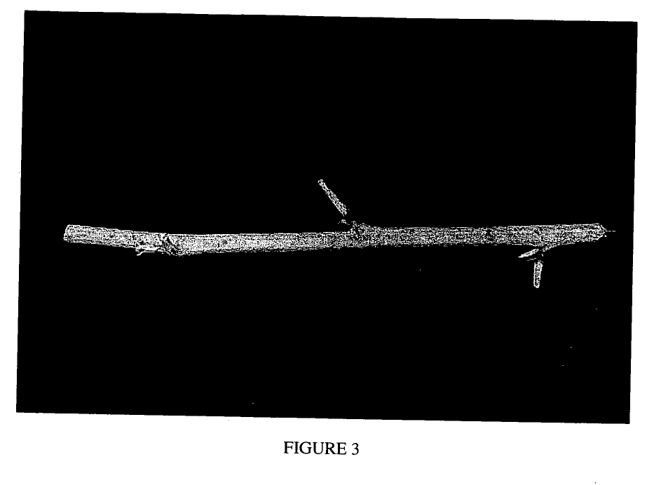

### 5. DETAILED BOTANICAL DESCRIPTION

The following detailed description of the new raspberry cultivar, ‘Driscoll Pacifica’, is based upon recorded observation of plants and fruit grown between 2003 and 2005 in Watsonville, Calif., and is believed to apply to plants of the ‘Driscoll Pacifica’ cultivar grown in similar conditions of soil and climate elsewhere.

Throughout this specification, color names beginning with a small letter signify that the name of the color, as used in common speech, is aptly descriptive. Color data beginning with a capital letter and followed by an alphanumeric code indicate the most similar color designations as provided by The Royal Horticultural Society (RHS) Colour Chart published by The Royal Horticultural Society of London, England. Color designations, color descriptions, and other phenotypical descriptions may deviate from the stated values and descriptions depending upon variation in environmental, seasonal, climatic and cultural conditions.

Table 1 provides information on the plant and fruit characteristics of the new raspberry cultivar ‘Driscoll Pacifica’ compared with characteristics of the unpatented raspberry cultivar ‘Heritage’. Observations of the cultivars were taken in comparisons under similar conditions.

The new variety is particularly characterized and distinguished from other cultivars by its fruit firmness, consistent fruit structure and high yield. The fruit color of ‘Driscoll Pacifica’ is a medium red at harvest and darkens very little after harvest. Fruit of ‘Driscoll Pacifica’ separates easily from the receptacle and is of excellent firmness at harvest. The fruit of ‘Driscoll Pacifica’ is consistently large throughout the harvest period.

The primocane and floricane yields of ‘Driscoll Pacifica’ are high relative to the variety ‘Heritage’. ‘Driscoll Pacifica’ is distinguishable from its pollen parent, selection ‘Q471.6’, by having better flavored fruit and by having larger fruit from its seed parent, selection ‘N234.1’.

### 5.1 Disease ABD Stress Resistance

Resistance is unknown to powdery mildew and root rots. Cold tolerance of the new cultivar has not been established. Post harvest fruit rot resistance is good in comparison over many selections and varieties.

TABLE 1
PLANT CHARACTERISTICS OF ‘DRISCOLL PACIFICA’

| | Driscoll Pacifica | Heritage |
|---|---|---|
| GENERAL | | |
| Plant Size | Med | Large |
| Growth habit | Erect | Erect |
| Productivity | High | Medium |
| Self-fruitfulness | Self-fruitful | Self-Fruitful |
| Time of bud burst | Late | Late |
| Primocane fruiting | | |
| Percent of cane | 40-60 | 20-40 |
| Length flowering as | | |
| Primocane | | |
| Percent of total yield | 58 | 53 |
| Primocanes | | |
| Number of young shoots | Many | Medium |
| Young shoot pigmentation | Medium | Medium |
| Length (cm) | 173 | 195 |
| Time of shoot emergence | Medium | Medium |
| Glaucosity (waxy bloom) | Absent or weak | Weak |
| Strength | Medium | Medium |
| Cane cross section | Rounded to | Rounded to |
| (from mid cane of primocane) | angular | angular |
| Dormant cane color | 165-B | 166-B |
| Prickles | | |
| Pigmentation | Green to brown | Greenish-brown |
| | green | to green |
| Density on young shoots | Sparse | Dense |
| Attitude of tip | Downward | Downward |
| Size | Medium | Medium |
| Size: Length (mm) (base | 0.8 | 2 |
| to tip at 1 m height at | | |
| end of harvest) | | |
| Texture | Soft | Heavy |
| Presence and distribution | Present, regularly | Present, regularly |
| of petioles | distributed | distributed |
| Pubescence on canes | Absent | Absent |
| Internodal distance (cm) | 5.5 | 4.3 |
| (at central ⅓ of cane) | | |
| LEAVES | | |
| Color | | |
| Face | 147-A | 147-A |
| Underside | 148-B | 148-C |
| Relief between veins | Medium | Weak |
| Glossiness | Medium | Medium |
| Petiole | | |
| Length (cm) | 5.1 | 7.0 |
| Pigmentation of upper | Lightly | Lightly |
| surface | | |
| Pigmentation of | Unpigmented | Unpigmented |
| underside | | |
| Stipule orientation | Clasping | Erect |
| Arrangement | Compound | Compound |
| Number of leaflets | Usually 3 | Sometimes 3, |
| | | sometimes 5 |
| Overlapping of lateral | Overlapping | Free to |
| leaflets | | touching |
| Lateral leaflet: length | Very short | Very short |
| to stalklet (lower pair) | | |
| Terminal leaflet | | |
| Length (cm) | 12.9 | 14.7 |
| Width (cm) | 9.3 | 8.8 |
| Shape | Ovate | Ovate |
| Tip | Acuminate | Acuminate |
| Base | Cordate | Rounded |
| Margin | Doubly serrate | Doubly serrate |
| Lateral leaflets | | |
| (basal pair) | | |
| Length (cm) | 9.4 | 13.7 |
| Width | 6.6 | 7.8 |
| Orientation | Opposite | Opposite |
| Shape | Ovate | Ovate |
| Tip | Acuminate | Acuminate |
| Base | Rounded | Oblique |
| Margin | Doubly serrate | Doubly serrate |
| Rachis length between | 3.4 | 4.6 |
| terminal leaflet and | | |
| adjacent lateral | | |
| leaflet (cm) | | |
| FLOWERS | | |
| Flowering period | | |
| Primocane | Late May-mid June | Early July through |
| | Through late | Early October |
| | September | |
| Floricane | Early April through | Mid April through |
| | Mid June | Mid June |
| Flower size | Medium | Medium |
| Petal | | |
| Length (cm) | 0.9 | 0.7 |
| Width (cm) | 0.4 | 0.3 |
| Pedicel | | |
| Coloration | Medium | Present, strong |
| | | Intensity |
| Length | Medium | Short to medium |
| FRUIT | | |
| Harvest Season | | |
| Primocane | Late June-late | Early August-early |
| | Oct | November |
| Floricane | Early April-mid | Late May-mid |
| | July | July |
| Color | Medium Red | Medium Red |
| Immature | 181-B | 180-A |
| Maturing | 184-A | 185-B |
| Mature fruit | 187-A | 185-A |
| Glossiness | Weak | Medium |
| Shape | Ovate | Ovate |
| Dimensions | | |
| Size | Medium | Small |
| Length (mm) | 25 | 19 |
| Width (mm) | 21 | 19 |
| Length:width | 1.2 | 1.0 |
| Weight (g/Fruit) | | |
| Primocane | 5.0 | 3.4 |
| Floricane | 4.6 | 2.6 |
| Soluble Solids (%) | 10.3 | 10.5 |
| Titratable acidity | 1.66 | 1.50 |
| (% as citric acid) | | |
| Seeds | | |
| Weight (mg/seed) | 1.42 | 1.62 |
| Number druplets/fruit | 116 | 75 |
| Adherence to plug (1-9) | Medium | Medium |
| Firmness | Med firm to firm | Firm |
| Yield | High | Medium |

What is claimed:

1. A new and distinctive cultivar of raspberry plant, substantially as shown and described.
